(19) United States
(12) Patent Application Publication
(10) Pub. No.: US 2005/0084465 A1
(43) Pub. Date: Apr. 21, 2005

(54) Scrub shampoo and pre-shampoo scrub cleanser

(76) Inventor: Brent Alan Baxter, Castro Valley, CA (US)

Correspondence Address: BRENT ALAN BAXTER, 5209 SEAVIEW AVE., CASTRO VALLEY, CA, 94546, US

(21) Appl. No.: 10/903,734

(22) Filed: Jul. 31, 2004

Related U.S. Application Data
(60) Provisional application No. 60/491,944, filed on Aug. 1, 2003.

(51) Int. Cl. A61K 7/06; A61K 7/11

(52) U.S. Cl. 424/70.1; 424/70.14

(57) ABSTRACT

This invention demonstrates a concentrated mixture of an exfoliating scrub shampoo, a purifying masque and a soothing, invigorating which both cleanse and stimulate the scalp for the express intent of promoting healthy hair growth and preventing hair loss. This scalp scrub mixture includes scrubbing agents, a purifying masque, binding ingredients and/or medication or soothing and nutrients for use on the scalp. This scrub cleanser for the scalp which feels good to use such as providing a massaging feeling, is invigorating, improves circulation and uses non-irritating ingredients which are hypoallergenic and noncomedogenic. This invention also demonstrates an exfoliating scalp scrub cleanser shampoo or pre-shampoo scrub cleanser for use before cleansing with other hair regiments.

### BACKGROUND OF THE INVENTION

1. Field of the Invention

This invention relates to the field of personal care especially concerning the are of skin care products.

Specifically, the present invention relates to a scrub cleansing shampoo that is used to exfoliate and deep clean the scalp skin and massage the scalp for the express intent of feeling good, promoting scalp circulation and promoting vigorous hair growth.

2. Prior Art

Skin care as part of a regimen of personal care has long been important to many people. This is especially true of facial skin care. Cleansers are designed to clean the skin, moisturizers are designed to soften, and exfoliators that attempt to remove dead skin. The number of skin care products is astronomical supporting the popularity and concern for skin care. However, most cleansers and skin care products that exfoliate are used for the face and body. Few products are designed to exfoliate and deep cleanse the scalp.

Cleansers are used to remove skin oils, dirt and other foreign material from the skin. Shampoo is a liquid-based medium that designed to cleanse the scalp. There is a need to improve growth of new skin below the dead skin layers in the scalp.

Often, an exfoliator is used on non-scalp regions in an attempt to remove dead skin cells exposing the living layers. This provides a smoother softer more youthful appearance, and allowing the living layers to grow and stay healthy. At the present time, exfoliating creams use an abrasive compound in a scrubbing motion to remove dead skin cells from the face. There is a need for scalp safe exfoliating compounds to be used on the scalp to invigorate scalp and promote healthy hair growth.

Accordingly, it is an object of the present invention to provide a new and effective exfoliating cream expressly for the use on the scalp.

Another object of the present invention is to provide an exfoliating cream that is appropriate for use with the scalp skin.

Still another object of the present invention is to provide an exfoliating cream that does not irritate the scalp skin.

Yet still another object of the present invention is to provide a skin cream that will promote the growth of new skin layers in the scalp.

### SUMMARY OF THE INVENTION

This invention demonstrates a concentrated mixture of an exfoliating scrub shampoo, a purifying masque and a soothing, invigorating which both cleanse and stimulate the scalp for the express intent of promoting healthy hair growth and preventing hair loss. This scalp scrub mixture includes scrubbing agents, a purifying masque, binding ingredients and/or medication or soothing and nutrients for use on the scalp. This scrub cleanser for the scalp which feels good to use such as providing a massaging feeling, is invigorating, improves circulation and uses non-irritating ingredients which are hypoallergenic and noncomedogenic. This invention also demonstrates an exfoliating scalp scrub cleanser shampoo or pre-shampoo scrub cleanser for use before cleansing with other hair regiments.

### DETAILED DESCRIPTION OF THE PREFERRED EMBODIMENT

This invention is created using a novel exfoliating composition for topical application to scalp skin tissue. The composition includes a combination of a scalp safe exfoliating materials mixed with a binding and purifying ingredient to promote healthy hair growth and prevent hair loss.

The preferred composition of this scrub shampoo is intended to use a blend of botanical extracts, natural and manmade scrubbing complex which exfoliates the scalp and ingredients that purify a 1a a cleanser/masque that help purify and condition scalp skin. The preferred scrub shampoo should be soap free, oil-free, hypoallergenic and noncomedogenic using natural ingredients and other ingredients to provide a thorough massaging but gentle enough cleansing for the scalp. It does this through a combination of deep pore cleansing that draws out imbedded excess oil and dirt.

1. A cleansing agent composition for topical application to scalp skin tissue for exfoliating and deep cleansing scalp skin tissue to invigorate and massage the scalp and promote more scalp circulation and to promote hair growth and inhibit hair loss:
a scrub cleansing agent containing non irritating scrubbing compounds of natural or manmade products and other substances typically included in shampoos and scrub cleansers; and
masque like cleansing agents made of botanical extracts or other materials designed for skin purification, cleansing, to remove oil, dirt and dead skin cells and for conditioning; and
soothing, or invigorating ingredients, and binding ingredients and, or medications and, or nutrients

2. A composition as claimed in claim 1 wherein said scrubbing compounds are micro beads.

3. A composition as claimed in claim 1 wherein said scrubbing compounds are finely crushed organic matter.

4. A composition as claimed in claim 1 wherein said scrubbing compounds baking soda.

5. A composition as claimed in claim 1 wherein said scrubbing compounds are soy extracts.

6. A composition as claimed in claim 1 wherein said binding ingredient is a glycerin.

7. A composition as claimed in claim 1 wherein said binding ingredient are aluminum silicates.

8. A composition as claimed in claim 7 wherein said aluminum silicate is kaolin.

9. A composition as claimed in claim 7 wherein said aluminum silicate is bentonite.

10. A method for use as a pre-treatment stimulant and cleanser component in the treatment of male pattern hair loss, comprising applying to the scalp a low irritability preparation comprising a scrub cleansing cleansing agent containing scrubbing compounds and one or more exfoliating ingredients including aluminum silicates.

11. A method for use as a pre-treatment stimulant and cleanser component to be used in a cleaning regiment promoting vigorous and healthy hair growth comprising applying to the scalp a low irritability preparation comprising a scrub cleansing cleansing agent containing scrubbing compounds and one or more exfoliating ingredients including aluminum silicates.

12. A method for massaging, invigorating and promoting scalp circulation comprising applying to the scalp a low irritability preparation comprising a scrub cleansing agent containing scrubbing compounds and one or more exfoliating ingredients including aluminum silicates.

13. A method designed to be used with other products as a pre-product use deep cleaner which opens up pores and deep cleans scalp for improved product performance of items such as shampoos and hair conditioners, or medications which are added afterward.
